(19) United States
(12) Patent Application Publication
(10) Pub. No.: US 2026/0326106 A1
(43) Pub. Date: Oct. 22, 2026

(54) UNIVERSAL DETECTION TEST TUBE BASED ON MICRO-MAGNETIC CONTROL TECHNOLOGY AND DETECTION METHOD

(71) Applicant: HEFEI DAHUI GENE TECHNOLOGY CO., LTD, Hefei (CN)

(72) Inventors: Zhizhen Xu, Hefei (CN); Dawei Fang, Hefei (CN); Nan Cheng, Hefei (CN)

(21) Appl. No.: 19/691,585

(22) Filed: May 29, 2026

Related U.S. Application Data
(63) Continuation of application No. PCT/CN2024/088477, filed on Apr. 18, 2024.

(30) Foreign Application Priority Data
Dec. 20, 2023 (CN) 202311754018.6

(51) Int. Cl. B01L 3/00; B01F 25/31; B01F 27/90; C12Q 1/6806

(52) U.S. Cl. CPC B01L 3/50825; B01F 25/31; B01F 27/90; C12Q 1/6806; B01L 2200/0689; B01L 2200/10; B01L 2200/16; B01L 2300/041

(57) ABSTRACT

A universal detection test tube based on micro-magnetic control technology and a detection method. The universal detection test tube includes a micro-magnetic control tube and a sealed transmission mechanism, the sealed transmission mechanism is arranged at a top of the micro-magnetic control tube, and a reaction chamber is arranged at a bottom of the micro-magnetic control tube. The sealed transmission mechanism includes a top cover. The top cover is provided with a first layer of threads and a second layer of threads, and a sealed transmission shaft is arranged within the top cover. A top clamping joint is arranged on a top of the sealed transmission shaft, and a supporting ring is arranged below the sealed transmission shaft. A bottom clamping joint is arranged below the supporting ring.

### CROSS REFERENCE TO RELATED APPLICATIONS

The present application is a Continuation Application of PCT Application No. PCT/CN2024/088477 filed on Apr. 18, 2024, which claims the benefit of Chinese Patent Application No. 202311754018.6 filed on Dec. 20, 2023. All the above are hereby incorporated by reference in their entirety.

### TECHNICAL FIELD

The present disclosure relates to the field of detection technology, and in particular to a universal detection test tube based on micro-magnetic control technology and a detection method.

### BACKGROUND

The principle of nucleic acid extraction by magnetic beads is based on the same principle as that of silica membrane centrifugal columns. By applying nanotechnology to modify and surface-treat a surface of superparamagnetic nanoparticles, superparamagnetic silica nanomagnetic beads are prepared. The magnetic beads can specifically identify and efficiently bind to nucleic acid molecules at a microscopic interface. By utilizing the superparamagnetism of silica-coated nanomagnetic microspheres, DNA and RNA can be separated from samples such as blood, animal tissues, food, and pathogenic microorganisms under an action of Chaotropic salts (such as guanidine hydrochloride, guanidine isothiocyanate) and external magnetic fields, which can be applied in many fields such as clinical disease diagnosis, blood transfusion safety, forensic identification, environmental microbial detection, food safety detection, and molecular biology research.

In modern medical detection, the testing items of various samples (such as sputum, blood, cerebrospinal fluid, pleural effusion, body fluids, pathological sections, etc.) need to go through processes such as pre-separation, mixing, stirring, temperature regulation, cleaning, and detection. However, there is no universal test tube that can process a variety of different samples for different tests based on existing technologies and methods.

For example, nucleic acid amplification and detection technology is a process in which nucleic acid fragments are amplified exponentially by providing conditions for in vitro amplification, fluorescent dyes or fluorescent markers are added during the nucleic acid amplification process, a strength of the fluorescent signal is detected by using optical instruments, to obtain the nucleic acid amplification results by processing and analyzing the fluorescent signal.

However, the test tubes for nucleic acid extraction and amplification in the art have the problem of poor sealing, which may easily cause contamination of sample nucleic acids. In addition, the nucleic acid extraction and amplification test in the art have a problem of insufficient reaction.

Moreover, in the art, nucleic acid extraction and nucleic acid amplification are two separate steps. The two steps in current normal processes are carried out separately, requiring two sets of instruments and reagents—one set for nucleic acid extraction (including extraction tubes and extraction instruments) and another set for nucleic acid amplification (including amplification tubes and amplification instruments).

### SUMMARY

The present disclosure provides a universal detection test tube based on micro-magnetic control technology and a detection method, which solves the above technical problems by means of a sealed transmission mechanism.

In one aspect of the present disclosure, provided is a universal detection test tube based on micro-magnetic control technology, including a micro-magnetic control tube and a sealed transmission mechanism, where the sealed transmission mechanism is arranged at a top of the micro-magnetic control tube, and a reaction chamber is arranged at a bottom of the micro-magnetic control tube;
the sealed transmission mechanism includes a top cover, the top cover is arranged on the top of the micro-magnetic control tube, the top cover is provided with a first layer of threads configured to connect to an internal seal groove and a second layer of threads configured to connect to an opening of the micro-magnetic control tube, a sealed transmission shaft is arranged within the top cover, a top clamping joint is arranged on a top of the sealed transmission shaft, a supporting ring with an outer diameter larger than an outer diameter of the sealed transmission shaft is arranged below the sealed transmission shaft, a bottom clamping joint with an outer diameter smaller than the outer diameter of the supporting ring is arranged below the supporting ring, the sealed transmission shaft, the supporting ring, the top clamping joint, and the bottom clamping joint are integrally formed as a single piece;
a liquid injection and stirring mechanism is arranged below the sealed transmission mechanism, and the liquid injection and stirring mechanism is configured to perform liquid injection and stirring; a preset mechanism is arranged on an outer side of the liquid injection and stirring mechanism, and the preset mechanism is configured to pre-store a solid or a liquid required for a reaction;
a sealed liquid injection mechanism and a reaction mechanism are arranged below the liquid injection and stirring mechanism;
the sealed liquid injection mechanism is configured to isolate a reagent in the micro-magnetic control tube from a reagent in the reaction mechanism; the reaction mechanism is configured to clean a nucleic acid to be detected; and the reaction chamber is configured to perform a reaction of the nucleic acid and a reaction liquid.

In a possible embodiment, a circular first through-hole is provided in a middle of the top cover, the first through-hole is provided with a circular groove formed horizontally, the supporting ring is movably connected to the circular groove, and the supporting ring rotates within the circular groove; a circular second through-hole is provided in a middle of the internal seal groove, and the internal seal groove is provided with threads configured to connect to the first layer of threads of the top cover.

In a possible embodiment, the top clamping joint is connected to an external motor drive mechanism, and the top clamping joint is rotated driven by the motor drive mechanism.

In a possible embodiment, the liquid injection and stirring mechanism includes a liquid injection hollow rod, a top clamping groove, and a double-layer stirring blade, an upper end of the liquid injection hollow rod is clamped with the bottom clamping joint, the double-layer stirring blade is threadedly fixedly connected to a lower end of the liquid injection hollow rod, and the double-layer stirring blade is located above the sealed liquid injection mechanism.

In a possible embodiment, the preset mechanism includes a preset cover and a preset groove, a circular fourth through-hole is provided in a middle of the preset cover, a cylindrical hole is provided in a middle of the preset groove, and the sealed transmission shaft passes through the cylindrical hole and the fourth through-hole.

In a possible embodiment, the preset groove is divided into a plurality of sub-grooves by a plurality of partitions in a vertical direction, a bottom of the preset cover is provided with a plurality of slots at positions corresponding to the plurality of partitions that are in the preset groove, and a bottom of each of the plurality of sub-grooves of the preset groove is designed as a slope opening.

In a possible embodiment, the sealed liquid injection mechanism includes a sealing plate and a liquid injection plate, the liquid injection plate is provided with a plurality of circular holes in communication with respective chambers of the reaction mechanism, a lower side of the sealing plate is provided with a plurality of cylindrical protrusions corresponding to the plurality of circular holes, and the plurality of cylindrical protrusions are configured to seal the plurality of circular holes.

In a possible embodiment, the reaction mechanism includes a first communication chamber, a first isolation chamber, a cleaning chamber, a second isolation chamber, and a second communication chamber, all of which are arranged circumferentially the second communication chamber is in communication with the reaction chamber, and the reaction chamber is a cone that is wide at a top and narrow at a bottom.

In another aspect, provided is a detection method involving nucleic acid stirring, extraction, and amplification, carried out using the universal detection test tube based on micro-magnetic control technology, and the detection method includes:
opening a cover of the micro-magnetic control tube, adding a sample into a space between the preset mechanism and the sealed liquid injection mechanism in the micro-magnetic control tube;
sealing the micro-magnetic control tube by means of the top cover, the sealed transmission shaft, and the internal seal groove;
rotating the sealed transmission shaft manually or by a motor drive;
mixing a liquid containing a magnetic bead with the sample within the preset groove, melting a paraffin wax at a bottom of the preset groove by heating, allowing a liquid at the bottom of the preset groove to flow into a middle portion of the micro-magnetic control tube;
inserting the micro-magnetic control tube into a shaftless ball gear, allowing the sample to raise temperature thereof and to be lysed, rotating an external magnet repeatedly to drive the magnetic bead inside the micro-magnetic control tube to move, where a nucleic acid released by lysis binds to the magnetic bead, and under an action of the external magnet, the magnetic bead sequentially passes through a first communication chamber, a first isolation chamber, a cleaning chamber, a second isolation chamber, and a second communication chamber, thereby completing cleaning;
melting a paraffin wax at an upper portion of the reaction chamber by temperature control, allowing the magnetic bead carrying the nucleic acid to enter the reaction chamber; and
separating the magnetic bead from the nucleic acid under an action of a reagent inside the reaction chamber, allowing the magnetic bead to a bottom of the reaction chamber, where the nucleic acid reacts with the reaction liquid in the reaction chamber, and a detection instrument collects fluorescence from a side of the reaction chamber.

In a possible embodiment, the detection method further includes following steps before the detection:
adding the reaction liquid, the paraffin wax and a biological reagent into the reaction chamber in sequence;
adding silicone oil into the first isolation chamber and the second isolation chamber of the reaction mechanism, and adding a cleaning liquid into the cleaning chamber;
preplacing a solidified paraffin wax at a bottom of each of a plurality of sub-grooves of the preset groove; and
adding the liquid containing the magnetic bead onto the solidified paraffin wax at a bottom of one of the plurality of sub-grooves of the preset groove.

Compared with the prior art, the present disclosure has the following beneficial effects.

For a universal test tube based on micro-magnetic control technology and a detection method in one embodiment of the present disclosure, reactions occur inside a micro-magnetic control tube, and a screw seal is adopted at the top for better sealing effect. A sealed transmission mechanism possesses both sealing and stirring functions, which can reduce a risk of sample nucleic acid contamination and ensure sufficient reaction through stirring. A paraffin wax is injected into an inner wall of an internal seal groove to provide secondary sealing, further improving the sealing effect.

Moreover, a liquid injection hollow rod of a liquid injection and stirring mechanism can facilitate an addition of a sample liquid into an interior of the micro-magnetic control tube, and the reaction liquid can be stirred more evenly by a double-layer stirring blade.

Any number of sub-grooves of the preset groove may be provided in a preset mechanism, which serve to pre-store various reagents and substances, and keep them isolated before use. A sealed liquid injection mechanism facilitates the injection of various reagents and substances into a reaction groove and provides sealing. Various grooves inside the reaction mechanism can be filled with different liquids, and driven by magnetic beads, the liquids can move among the different chambers to achieve desired effects. Finally, the required substances move together with the magnetic beads into a reaction chamber for mixing. Inside the reaction chamber, a series of specific reactions occur under specific conditions. The inner bottom of each sub-groove of the preset groove adopts a sloped opening design to facilitate an outflow of reagents from the preset groove into the interior of the micro-magnetic control tube. Through the coordination of the preset mechanism and the reaction mechanism, it can further process a variety of different samples for different detection applications.

By using the universal detection test tube based on micro-magnetic control technology according to the embodiments of the present disclosure, two steps are integrated using the magnetic bead method, which saves operational steps, time, space, and cost. Nucleic acids are transferred in a sealed environment, thereby preventing aerosol contamination.

### BRIEF DESCRIPTION OF THE DRAWINGS

FIG. 1 shows a cutaway view from a first perspective of a universal detection test tube based on micro-magnetic control technology according to an embodiment of the present disclosure.

FIG. 2 shows a cutaway view from a second perspective of a universal detection test tube based on micro-magnetic control technology according to an embodiment of the present disclosure.

FIG. 3 shows a partial cutaway view of a universal detection test tube based on micro-magnetic control technology according to an embodiment of the present disclosure.

FIG. 4 is a schematic diagram showing a sealed liquid injection mechanism, a reaction mechanism and a reaction chamber according to an embodiment of the present disclosure.

FIG. 5 shows an exploded view of a universal detection test tube based on micro-magnetic control technology according to an embodiment of the present disclosure.

FIG. 6 shows a schematic diagram of a universal detection test tube based on micro-magnetic control technology according to an embodiment of the present disclosure.

FIG. 7 is a partial cutaway view of a preset mechanism according to an embodiment of the present disclosure.

In the drawings: 1, micro-magnetic control tube; 2, sealed transmission mechanism; 21, top cover; 22, sealed transmission shaft; 23, supporting ring; 24, top clamping joint; 25, bottom clamping joint; 26, internal seal groove; 3, liquid injection and stirring mechanism; 31. liquid injection hollow rod; 32. top clamping groove; 33. double-layer stirring blade; 4. preset mechanism; 41. preset cover; 42. preset groove; 43. partition; 44. fourth through-hole; 45. cylindrical hole; 5. sealed liquid injection mechanism; 51. sealing plate; 52. liquid injection plate; 53. circular hole; 54. cylindrical protrusion; 6. reaction mechanism; 61. first communication chamber; 62. first isolation chamber; 63. cleaning chamber; 64. second isolation chamber; 65. second communication chamber; 7. reaction chamber.

### DETAILED DESCRIPTION

Various exemplary embodiments, features and aspects of the present disclosure will be described in detail below with reference to the accompanying drawings. The same reference numerals in the accompanying drawings represent elements with the same or similar functions. Although various aspects of the embodiments are shown in the accompanying drawings, the drawings are not necessarily drawn to scale unless otherwise specified.

The term “exemplary” is used exclusively herein to mean “serving as an example, example, or illustration.” Any embodiment described herein as “exemplary” is not necessarily to be construed as preferred or advantageous over other embodiments.

In addition, in order to better illustrate the present disclosure, numerous specific details are given in the following specific embodiments. It should be understood by those skilled in the art that the present disclosure can also be implemented without certain specific details. In some examples, methods, means, components and circuits well known to those skilled in the art are not described in detail in order to highlight the subject matter of the present disclosure.

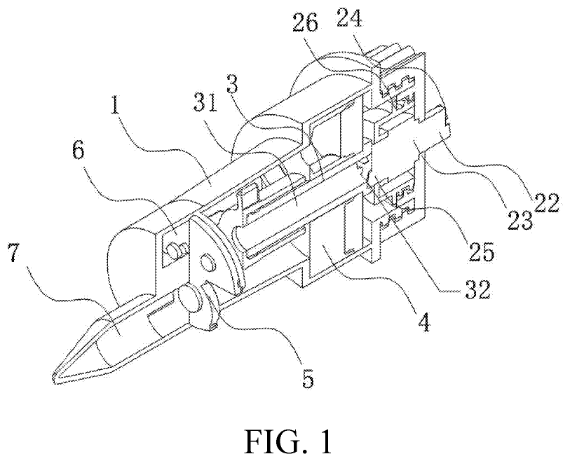

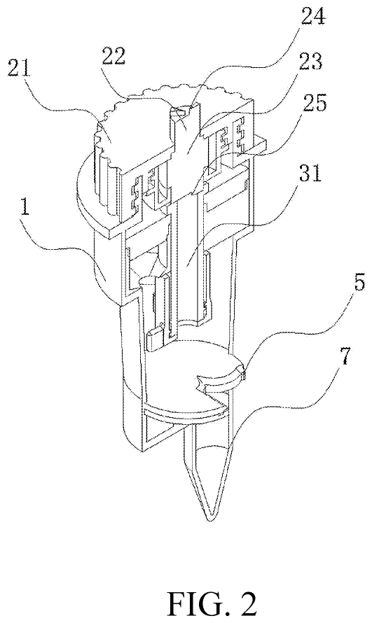

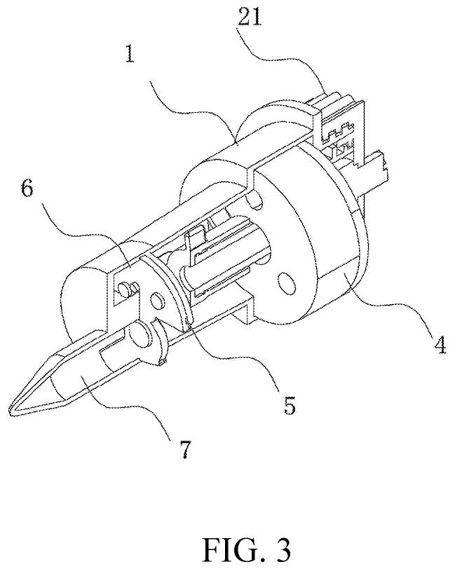

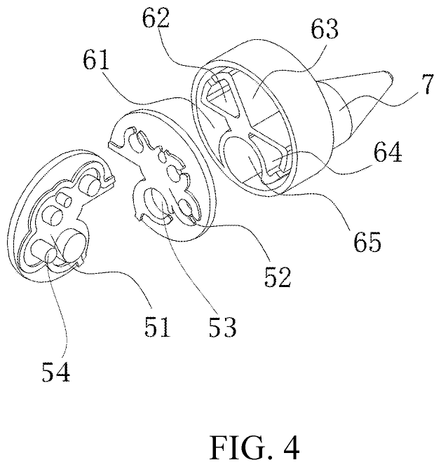

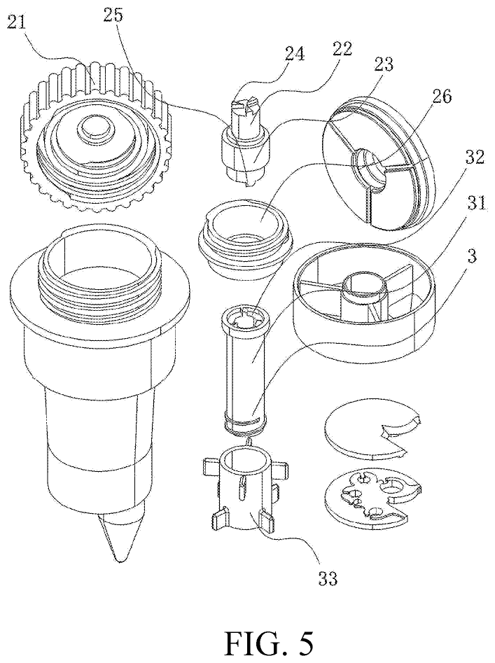

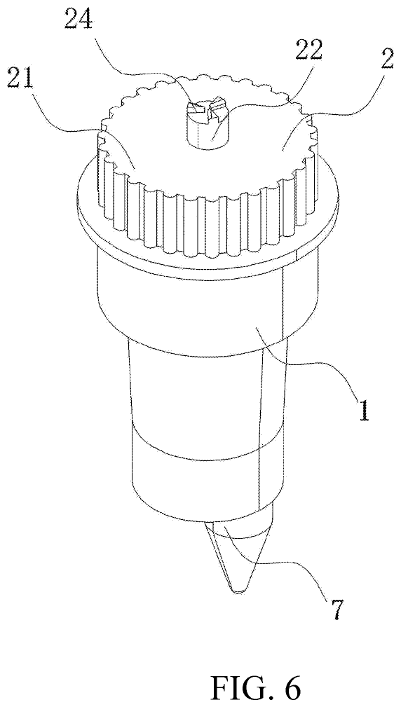

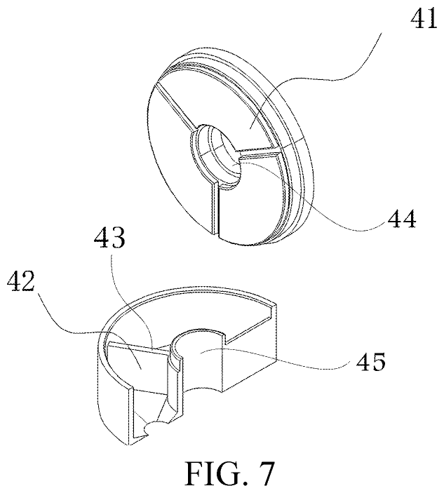

Referring to FIGS. 1-7, a universal detection test tube based on micro-magnetic control technology of the present disclosure includes a micro-magnetic control tube 1, a sealed transmission mechanism 2, a liquid injection and stirring mechanism 3, a preset mechanism 4, a sealed liquid injection mechanism 5, a reaction mechanism 6, and a reaction chamber 7.

The sealed transmission mechanism 2 includes a top cover 21, a sealed transmission shaft 22, a supporting ring 23, a top clamping joint 24, a bottom clamping joint 25, and an internal seal groove 26. The top cover 21 is provided with a first layer of threads configured to connect to the internal seal groove 26 and a second layer of threads configured to connect to an opening of the micro-magnetic control tube 1. The top clamping joint 24 is arranged on a top of the sealed transmission shaft 22. The supporting ring 23 is arranged in a middle portion of the sealed transmission shaft 22, and the middle portion refers to a position of the sealed transmission shaft 22 that is not at two ends thereof. An outer diameter of the supporting ring 23 is larger than an outer diameter of the sealed transmission shaft 22. The sealed transmission shaft 22, the supporting ring 23, the top clamping joint 24, and the bottom clamping joint 25 are integrally formed as a single piece.

The liquid injection and stirring mechanism 3 includes a liquid injection hollow rod 31, a top clamping groove 32, and a double-layer stirring blade 33, where an upper end of the liquid injection hollow rod 31 is clamped with the bottom clamping joint 25, and the double-layer stirring blade 33 is threadedly fixedly connected to a lower end of the liquid injection hollow rod 31.

A circular first through-hole is provided in a middle of the top cover 21. The first through-hole is provided with a circular groove formed horizontally. The supporting ring 23 is movably connected to the circular groove. The supporting ring 23 can rotate in the circular groove.

A circular second through-hole is provided in a middle of the internal seal groove 26, and the internal seal groove 26 is provided with threads configured to connect to the first layer of threads of the top cover 21.

The preset mechanism 4 is configured to pre-store a solid or a liquid used for the reaction.

The sealed liquid injection mechanism 5 is configured to isolate a reagent in the micro-magnetic control tube 1 from a reagent in the reaction mechanism 6.

The reaction mechanism 6 is configured to clean a nucleic acid to be detected.

The reaction chamber 7 is configured to perform a reaction between the nucleic acid and the reaction liquid.

After an installation is completed, the double-layer stirring blade 33 is located above the sealed liquid injection mechanism 5.

For a universal test tube based on micro-magnetic control technology and a detection method in one embodiment of the present disclosure, reactions occur inside a micro-magnetic control tube, and a screw seal is adopted at the top for better sealing effect. A sealed transmission mechanism possesses both sealing and stirring functions, which can reduce a risk of sample nucleic acid contamination and ensure sufficient reaction through stirring. A paraffin wax is injected into an inner wall of an internal seal groove to provide secondary sealing, further improving the sealing effect. Moreover, a liquid injection hollow rod of a liquid injection and stirring mechanism can facilitate an addition of a sample liquid into an interior of the micro-magnetic control tube, and the reaction liquid can be stirred more evenly by a double-layer stirring blade.

Any number of sub-grooves of the preset groove may be provided in a preset mechanism, which serve to pre-store various reagents and substances, and keep them isolated before use. A sealed liquid injection mechanism facilitates the injection of various reagents and substances into a reaction groove and provides sealing. Various grooves inside the reaction mechanism can be filled with different liquids, and driven by magnetic beads, the liquids can move among the different chambers to achieve desired effects. Finally, the required substances move together with the magnetic beads into a reaction chamber for mixing. Inside the reaction chamber, a series of specific reactions occur under specific conditions. The inner bottom of each sub-groove of the preset groove adopts a sloped opening design to facilitate an outflow of reagents from the preset groove into the interior of the micro-magnetic control tube.

In a possible embodiment, the preset mechanism 4 includes a preset cover 41 and a preset groove 42.

The preset mechanism 4 includes a preset cover 41 and a preset groove 42. A circular fourth through-hole 44 is provided in a middle of the preset cover 41. A cylindrical hole 45 is provided in a middle of the preset groove 42. The sealed transmission shaft 22 passes through the cylindrical hole 45 and the fourth through-hole 44.

The preset groove 42 is divided into a plurality of sub-grooves by a plurality of partitions 43 in a vertical direction, a bottom of the preset cover 41 is provided with a plurality of slots at positions corresponding to the plurality of partitions 43 that are in the preset groove 42.

A bottom of each of the plurality of sub-grooves of the preset groove 42 is designed as a slope opening. Any number of sub-grooves of the preset groove 42 may be provided in a preset mechanism 4, which serve to pre-store various reagents and solid substances, and keep different liquids and solid substances isolated before use. The inner bottom of each sub-groove of the preset groove 42 adopts a sloped opening design to facilitate an outflow of reagents from the preset groove 42 into the interior of the micro-magnetic control tube 1.

The universal detection test tube based on micro-magnetic control technology and the detection method can also be applied to other technical fields such as chemiluminescence and chemiluminescence immunoassay, utilizing magnetic bead adsorption to achieve the same luminescence detection effect. Other sub-grooves of the preset groove (42) are reserved for adding other reagents that need to be isolated when used in chemiluminescence immunoassay and other methods.

In a possible embodiment, the sealed liquid injection mechanism 5 includes a sealing plate 51 and a liquid injection plate 52. The reaction mechanism 6 includes a first communication chamber 61, a first isolation chamber 62, a cleaning chamber 63, a second isolation chamber 64, and a second communication chamber 65.

The liquid injection plate 52 is provided with a plurality of circular holes 53 in communication with respective chambers of the reaction mechanism 6. A lower side of the sealing plate 51 is provided with a plurality of cylindrical protrusions 54 corresponding to the plurality of circular holes 53, and the plurality of cylindrical protrusions 54 are configured to seal the plurality of circular holes 53.

The second communication chamber 65 is in communication with the reaction chamber 7.

The reaction chamber 7 is a cone that is wide at a top and narrow at a bottom.

The sealed liquid injection mechanism 5 is configured to facilitate an injection of various reagents and substances into the reaction groove and to seal the reaction groove.

In a possible embodiment, the top clamping joint 24 is connected to an external motor drive mechanism, and the top clamping joint 24 is rotated driven by the motor drive mechanism.

Alternatively, the sealed transmission shaft 22 is rotated manually to allow the reaction in the test tube to proceed fully, for example, to accelerate a lysis reaction so that the sample is fully lysed.

The present disclosure discloses a detection method involving nucleic acid stirring, extraction, and amplification, carried out using the universal detection test tube based on micro-magnetic control technology. The detection method includes:
adding a reaction liquid, a paraffin wax and a biological reagent into the reaction chamber 7 in sequence;
adding silicone oil into the first isolation chamber 62 and the second isolation chamber 64 of the reaction mechanism 6, and adding a cleaning liquid into the cleaning chamber 63; where the silicone oil and the cleaning liquid are added for cleaning the magnetic bead;
preplacing a solidified paraffin wax at a bottom of each of a plurality of sub-grooves the preset groove 42; and
adding a liquid containing a magnetic bead onto the solidified paraffin wax at a bottom of one of the plurality of sub-grooves of the preset groove 42.

The above steps are for packaging the detection reagents of the micro-magnetic control tube.

A cover of the micro-magnetic control tube 1 is opened, and a tip of a pipette passes through the liquid injection hollow rod 31 and the double-layer stirring blade 33 to go deep into a space between the preset mechanism 4 and the sealed liquid injection mechanism 5 for adding a sample liquid. The micro-magnetic control tube 1 is sealed by the top cover 21, the sealed transmission shaft 22 and the internal seal groove 26.

The sealed transmission shaft 22 is rotated manually or by a motor drive.

In the preset groove 42, the liquid containing the magnetic bead is mixed with the test sample. The paraffin wax at the bottom of the preset groove 42 is melted by heating, and a liquid at the bottom of the preset groove 42 is allowed to flow into the middle portion of the micro-magnetic control tube 1.

The micro-magnetic control tube 1 is inserted into a shaftless ball gear, the test sample is heated to lysis, and an external magnet is repeatedly rotated to drive the movement of the magnetic bead inside the micro-magnetic control tube 1. The nucleic acid released by lysis binds to the magnetic bead. Under an action of the external magnet, the magnetic bead sequentially passes through the first communication chamber 61, the first isolation chamber 62, the cleaning chamber 63, the second isolation chamber 64, and the second communication chamber 65, thereby completing cleaning.

For example, the cleaning chamber 63 contains a cleaning liquid, which is used to clean the magnetic bead to remove impurities.

The paraffin wax at the upper portion of the reaction chamber 7 is melted by temperature control, and the magnetic bead carrying the nucleic acid is allowed to enter the reaction chamber 7.

Under an action of the reagent inside the reaction chamber 7, the magnetic bead is separated from the nucleic acid. The magnetic bead is settled to the bottom of the reaction chamber 7, and the nucleic acid reacts with the reagent inside the reaction chamber 7. Fluorescence is collected from a side of the reaction chamber 7 by a detection instrument.

An application example of using the micro-magnetic control tube is provided.

An inner bottom of each of the plurality of sub-grooves of the preset groove 42 is preset with a solidified paraffin wax in advance, and the liquid containing the magnetic bead is added onto the solidified paraffin wax at a bottom of one of the plurality of sub-grooves of the preset groove 42. Other liquids that need to be isolated and preset can be selectively added to other sub-grooves, and the preset cover 41 is closed to achieve a purpose of presetting.

When in use, the sealed transmission mechanism 2 is first unscrewed by rotating. A tip of a pipette is used for sucking the sample, and the tip passes through the liquid injection hollow rod 31 and the double-layer stirring blade 33 to go deep into a space between the preset mechanism 4 and the sealed liquid injection mechanism 5, so that the liquid sample is added into the interior of the micro-magnetic control tube 1. Afterwards, the sealed transmission mechanism 2 is closed by rotating, such that the bottom connecting joint 25 is clamped with the top clamping groove 32. Then, the micro-magnetic control tube 1 is placed into a detection instrument for detection.

During the reaction, the paraffin wax at the bottom of the preset groove 42 is melted by heating. Due to the sloped opening design of the inner bottom of the preset groove 42, the paraffin wax, the liquid containing the magnetic bead, and other preset liquids are facilitated to flow out of the preset groove 42 into the middle portion of the micro-magnetic control tube 1, where they are mixed with the liquid in the middle portion of the micro-magnetic control tube 1. By rotating the bottom connecting joint 25, the various liquids and the magnetic bead are more thoroughly mixed with the liquid in the middle portion of the micro-magnetic control tube 1. Meanwhile, under an action of an external magnet, the magnetic beads are gathered directly above the first communication chamber 61. The magnetic beads are moved sequentially along the first communication chamber 61, the first isolation chamber 62, the cleaning chamber 63, and the second isolation chamber 64 until they are moved to a position directly above the second communication chamber 65. Finally, the magnetic beads fall through the second communication chamber 65 into the interior of the reaction chamber 7 for reaction.

For a universal test tube based on micro-magnetic control technology and a detection method in one embodiment of the present disclosure, reactions occur inside a micro-magnetic control tube, and a screw seal is adopted at the top for better sealing effect. A sealed transmission mechanism possesses both sealing and stirring functions, which can reduce a risk of sample nucleic acid contamination and ensure sufficient reaction through stirring. A paraffin wax is injected into an inner wall of an internal seal groove to provide secondary sealing.

Any number of sub-grooves of the preset groove may be provided in a preset mechanism, which serve to pre-store various reagents and substances, and keep them isolated before use. A sealed liquid injection mechanism is configured to facilitate the injection of various reagents and substances into a reaction groove and provides sealing. Various grooves inside the reaction mechanism can be filled with different liquids, and driven by magnetic beads, the liquids can move among the different chambers to achieve desired effects. Finally, the required substances move together with the magnetic beads into a reaction chamber for mixing. Inside the reaction chamber, a series of specific reactions occur under specific conditions. The inner bottom of each sub-groove of the preset groove adopts a sloped opening design to facilitate an outflow of reagents from the preset groove into the interior of the micro-magnetic control tube.

The universal detection test tube based on micro-magnetic control technology and the method based on micro-magnetic control disclosed in the present disclosure, can achieve universal detection of multiple sample types (such as sputum, blood, cerebrospinal fluid, pleural and ascitic fluid, body fluids, pathological sections, etc.). Corresponding nucleic acid amplification detection technology and chemiluminescence immunoassay detection technology are merely specific embodiments of the detection methods that can be used with the universal detection test tube.

It should be noted that the foregoing embodiments of the method are described as a series of operation combinations for simplicity of description. However, it should be understood by those skilled in the art that the present disclosure is not limited by the described sequence of operations, as certain steps may be performed in other orders or simultaneously according to the present disclosure. Furthermore, it should be understood by those skilled in the art that the embodiments described in the description are optional embodiments, and the operations and modules involved are not necessarily required for the present disclosure.

In the above embodiments, the description of each embodiment has its own emphasis. For parts not described in detail in a particular embodiment, reference may be made to the relevant descriptions in other embodiments.

In the several embodiments provided in the present disclosure, it should be understood that the disclosed instruments may be carried out in other ways. For example, the instrument embodiments described above are merely illustrative. For instance, the division of units is merely a logical functional division, and other divisions may be used in actual implementation. For example, multiple units or components may be combined or integrated into another system, or some features may be ignored or not performed. In another aspect, the mutual coupling, direct coupling, or communication connections shown or discussed may be indirect couplings or communication connections implemented through some interfaces, devices, or units, and may be electrical or other forms.

The embodiments of the present disclosure have been introduced in detail above. Specific examples are used herein to illustrate the principles and implementations of the present disclosure. The description of the above embodiments is merely provided to assist in understanding the method and core ideas of the present disclosure. Meanwhile, for those skilled in the art, changes may be made to the specific implementations and application scopes according to the ideas of the present disclosure. In summary, the content of this description should not be construed as limiting the present disclosure.

Various aspects of the present disclosure are described herein with reference to flowcharts and/or block diagrams of methods, apparatuses (systems), and computer program products according to embodiments of the present disclosure. It will be understood that each block of the flowcharts and/or block diagrams, and combinations of blocks in the flowcharts and/or block diagrams, may be implemented by computer-readable program instructions.

The embodiments of the present disclosure have been described above, and the foregoing description is illustrative, not exhaustive, and is not limited to the disclosed embodiments. Many modifications and variations will be apparent to those skilled in the art without departing from the scope and spirit of the described embodiments. The terminology used herein is chosen to best explain the principles of the embodiments, practical applications, or technical improvements in the market, or to enable others skilled in the art to understand the embodiments disclosed herein.

What is claimed is:

1. A universal detection test tube based on micro-magnetic control technology, comprising a micro-magnetic control tube (1) and a sealed transmission mechanism (2), wherein the sealed transmission mechanism (2) is arranged at a top of the micro-magnetic control tube (1), and a reaction chamber (7) is arranged at a bottom of the micro-magnetic control tube (1);
the sealed transmission mechanism (2) comprises a top cover (21), the top cover (21) is arranged on the top of the micro-magnetic control tube (1), the top cover (21) is provided with a first layer of threads configured to connect to an internal seal groove (26) and a second layer of threads configured to connect to an opening of the micro-magnetic control tube (1), a sealed transmission shaft (22) is arranged within the top cover (21), a top clamping joint (24) is arranged on a top of the sealed transmission shaft (22), a supporting ring (23) with an outer diameter larger than an outer diameter of the sealed transmission shaft (22) is arranged below the sealed transmission shaft (22), a bottom clamping joint (25) with an outer diameter smaller than the outer diameter of the supporting ring (23) is arranged below the supporting ring (23), and the sealed transmission shaft (22), the supporting ring (23), the top clamping joint (24), and the bottom clamping joint (25) are integrally formed as a single piece;
a liquid injection and stirring mechanism (3) is arranged below the sealed transmission mechanism (2), and the liquid injection and stirring mechanism (3) is configured to perform liquid injection and stirring; a preset mechanism (4) is arranged on an outer side of the liquid injection and stirring mechanism (3), and the preset mechanism (4) is configured to pre-store a solid or a liquid required for a reaction;
a sealed liquid injection mechanism (5) and a reaction mechanism (6) are arranged below the liquid injection and stirring mechanism (3); and
the sealed liquid injection mechanism (5) is configured to isolate a reagent in the micro-magnetic control tube (1) from a reagent in the reaction mechanism (6); the reaction mechanism (6) is configured to clean a nucleic acid to be detected; and the reaction chamber (7) is configured to perform a reaction of the nucleic acid and a reaction liquid.

2. The universal detection test tube based on micro-magnetic control technology of claim 1, wherein a circular first through-hole is provided in a middle of the top cover (21), the first through-hole is provided with a circular groove formed horizontally, the supporting ring (23) is movably connected to the circular groove, and the supporting ring (23) rotates within the circular groove; a circular second through-hole is provided in a middle of the internal seal groove (26), and the internal seal groove (26) is provided with threads configured to connect to the first layer of threads of the top cover (21).

3. The universal detection test tube based on micro-magnetic control technology of claim 1, wherein the top clamping joint (24) is connected to an external motor drive mechanism, and the top clamping joint (24) is rotated driven by the motor drive mechanism.

4. The universal detection test tube based on micro-magnetic control technology of claim 1, wherein the liquid injection and stirring mechanism (3) comprises a liquid injection hollow rod (31), a top clamping groove (32) and a double-layer stirring blade (33), an upper end of the liquid injection hollow rod (31) is clamped with the bottom clamping joint (25), the double-layer stirring blade (33) is threadedly fixedly connected to a lower end of the liquid injection hollow rod (31), and the double-layer stirring blade (33) is located above the sealed liquid injection mechanism (5).

5. The universal detection test tube based on micro-magnetic control technology of claim 1, wherein the preset mechanism (4) comprises a preset cover (41) and a preset groove (42), a circular fourth through-hole (44) is provided in a middle of the preset cover (41), a cylindrical hole (45) is provided in a middle of the preset groove (42), and the sealed transmission shaft (22) passes through the cylindrical hole (45) and the fourth through-hole (44).

6. The universal detection test tube based on micro-magnetic control technology of claim 5, wherein the preset groove (42) is divided into a plurality of sub-grooves by a plurality of partitions (43) in a vertical direction, a bottom of the preset cover (41) is provided with a plurality of slots at positions corresponding to the plurality of partitions (43) that are in the preset groove (42), and a bottom of each of the plurality of sub-grooves of the preset groove (42) is designed as a slope opening.

7. The universal detection test tube based on micro-magnetic control technology of claim 1, wherein the sealed liquid injection mechanism (5) comprises a sealing plate (51) and a liquid injection plate (52), the liquid injection plate (52) is provided with a plurality of circular holes (53) in communication with respective chambers of the reaction mechanism

(6) a lower side of the sealing plate (51) is provided with a plurality of cylindrical protrusions (54) corresponding to the plurality of circular holes (53), and the plurality of cylindrical protrusions (54) are configured to seal the plurality of circular holes (53).

8. The universal detection test tube based on micro-magnetic control technology of claim 1, wherein the reaction mechanism (6) comprises a first communication chamber (61), a first isolation chamber (62), a cleaning chamber (63), a second isolation chamber (64), and a second communication chamber (65), all of which are arranged circumferentially, the second communication chamber (65) is in communication with the reaction chamber (7), and the reaction chamber (7) is a cone that is wide at a top and narrow at a bottom.

9. A detection method involving nucleic acid stirring, extraction, and amplification, carried out using the universal detection test tube based on micro-magnetic control technology of claim 1, and the detection method comprises:
opening a cover of the micro-magnetic control tube (1), adding a sample into a middle portion of the micro-magnetic control tube (1) by passing a tip of a pipette through the liquid injection hollow rod 31 and the double-layer stirring blade 33 to go deep into a space between the preset mechanism (4) and the sealed liquid injection mechanism (5) in the micro-magnetic control tube (1);
sealing the micro-magnetic control tube (1) by means of the top cover (21), the sealed transmission shaft (22), and the internal seal groove (26);
rotating the sealed transmission shaft (22) manually or by a motor drive;
melting a paraffin wax at a bottom of the preset groove (42) by heating, allowing a liquid containing a magnetic bead at the bottom of the preset groove (42) to flow into the middle portion of the micro-magnetic control tube (1), mixing the liquid containing the magnetic bead with the sample in the middle portion of the micro-magnetic control tube (1);
inserting the micro-magnetic control tube (1) into a shaftless ball gear, allowing the sample to raise temperature thereof and to be lysed, rotating an external magnet repeatedly to drive the magnetic bead inside the micro-magnetic control tube (1) to move, wherein a nucleic acid released by lysis binds to the magnetic bead, and under an action of the external magnet, the magnetic bead sequentially passes through a first communication chamber (61), a first isolation chamber (62), a cleaning chamber (63), a second isolation chamber (64), and a second communication chamber (65), thereby completing cleaning;
melting a paraffin wax at an upper portion of the reaction chamber (7) by temperature control, allowing the magnetic bead carrying the nucleic acid to enter the reaction chamber (7); and
separating the magnetic bead from the nucleic acid under an action of a reagent inside the reaction chamber (7), allowing the magnetic bead to a bottom of the reaction chamber (7), wherein the nucleic acid reacts with the reaction liquid in the reaction chamber (7), and a detection instrument collects fluorescence from a side of the reaction chamber (7).

10. The detection method involving nucleic acid stirring, extraction, and amplification of claim 9, wherein the detection method further comprises following steps before a detection:
adding the reagent and the paraffin wax into the reaction chamber (7) in sequence;
adding silicone oil into the first isolation chamber (62) and the second isolation chamber (64) of the reaction mechanism (6), and adding a cleaning liquid into the cleaning chamber (63);
preplacing a solidified paraffin wax at a bottom of each of a plurality of sub-grooves of the preset groove (42); and
adding the liquid containing the magnetic bead onto the solidified paraffin wax at a bottom of one of the plurality of sub-grooves of the preset groove (42).
